(19) United States
(12) Patent Application Publication
(10) Pub. No.: US 2005/0169876 A1
(43) Pub. Date: Aug. 4, 2005

(54) Composition for treating keratin material, process of making, uses thereof

(75) Inventor: Roland De La Mettrie, Le Vesinet (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/044,351

(22) Filed: Jan. 28, 2005

Related U.S. Application Data
(60) Provisional application No. 60/545,192, filed on Feb. 18, 2004.

(30) Foreign Application Priority Data
Jan. 29, 2004 (FR) 0400845

(51) Int. Cl. A61K 7/06; A61K 7/075; A61K 7/08

(52) U.S. Cl. 424/70.27

(57) ABSTRACT

The present invention relates to a process for preparing a composition useful for example for treating keratin materials, where a fluid containing at least steam, at a pressure ranging from 3 to 30 bar, is percolated through at least one silicone or non-silicone conditioning agent, in solid or pasty form, where the at least one silicone or non-silicone conditioning agent is chosen preferably from linear or branched fatty alcohols containing from 14 to 50 carbon atoms, linear or branched hydrocarbons, of mineral or synthetic origin, waxes, compounds of ceramide type, and organomodified or non-organomodified silicone gums or resins. The invention also relates to the compositions prepared, and uses thereof.

### REFERENCE TO PRIOR APPLICATIONS

This application claims priority to U.S. provisional application 60/545,192 filed Feb. 18, 2004, and to French patent application 0400845 filed Jan. 29, 2004, both incorporated herein by reference.

### FIELD OF THE INVENTION

The present invention relates to a process for preparing a (cosmetic) composition for the treatment of keratin materials, in particular the skin and human keratin fibres such as the hair, and also to a cosmetic process for treating keratin materials using this composition.

More particularly, the present invention relates to a process for preparing a (cosmetic) composition for treating keratin materials, wherein it comprises a step of percolating fluid comprising at least steam, at a pressure ranging from 3 to 30 bar, through at least one silicone or non-silicone conditioning agent, in solid or pasty form, chosen preferably from linear or branched fatty alcohols containing from 14 to 50 carbon atoms, linear or branched hydrocarbons, of mineral or synthetic origin, waxes, compounds of ceramide type, and organomodified or non-organomodified silicone gums or resins, the compositions prepared, and uses thereof.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

In cosmetics, it is always sought to improve the cosmetic properties of keratin materials, for example sensitized hair, i.e. hair that has been damaged or embrittled by the chemical action of atmospheric agents and/or hair treatments such as permanent-waving, dyeing or bleaching. Cosmetic treatment compositions comprising silicone or non-silicone conditioning agents, such as waxes, ceramides or silicones, intended to repair or limit the harmful or adverse effects produced by the various treatments or attacking factors to which keratin materials are more or less repeatedly subjected, may thus be applied to these materials. These conditioning agents may also improve the cosmetic properties of natural hair.

Conditioning agents such as waxes, ceramides or silicones give dry or wet hair, a styling and fixing effect, ease of disentangling, softness and/or smoothness.

These conditioning agents also give the skin cosmetic properties, such as good moisturization and good nourishment. In addition, waxes allow wrinkles to be smoothed and softened.

However, cosmetic treatment compositions containing such conditioning agents are generally aqueous compositions in which the agents must be dissolved. The lack of solubility of these compounds reduces the conditioning power of these compositions. In addition, this solubility criterion reduces the number of waxes, ceramides and silicones that can be used for the cosmetic treatment of keratin materials. This is particularly the case for compounds with a high melting point.

### DETAILED DESCRIPTION OF THE PREFERRED EMBODIMENTS

It has now been discovered, surprisingly, that by using a novel process for preparing a (cosmetic) composition for treating keratin materials, compositions that are more or less concentrated in conditioning agent(s) may be obtained in a very short time, for example of less than 2 minutes, according to need, especially without preserving agent, enabling the solubility problems outlined above to be overcome.

This process may be performed simply and is appropriate to the needs of the consumer. A fluid, the temperature of which is preferably greater than or equal to 30° C. is passed, under pressure, for a very short time, for example of less than 1 minute, through at least one conditioning agent in solid or pasty form, preferably solid and even more preferably pulverulent.

It also enables the use in anhydrous form of conditioning agents that are unstable in aqueous compositions either because they react with water or because they react in aqueous solution with compounds that do not react with them in an anhydrous composition.

The compositions prepared according to this process may have limited stability on storage, which is not a drawback in this case since the process leads to a ready-to-use composition intended to be used quickly after its preparation, for example within five minutes of preparation, especially after cooling to a temperature which is acceptable for keratin materials, preferably below 60° C., better still below 50° C. The composition may also be used up to one week or more after its preparation, depending on the rate of degradation of the conditioning agent used.

Given the very short preparation time, the cosmetic treatment compositions may be prepared “on demand” by mixing different cosmetically active compounds according to the desired cosmetic properties.

According to another embodiment, because the conditioning agents may be packaged in a ready-to-use device, it is not necessary to determine beforehand the concentrations of said agents in solution, which limits the measuring errors by the user.

In addition, the process according to the invention makes it possible to avoid the use of multi-compartment bottles, which makes the process particularly economical and safer for the user.

The composition thus obtained may be used alone or as a mixture with another composition.

A further advantage of this preparation process is the production of compositions that have better cosmetic properties. In particular, keratin fibres treated with a composition obtained via the process according to the invention have improved conditioning properties, especially in terms of disentangling, smoothness, sheen and soft feel, and the skin treated in this way has improved moisturization and smoothing of wrinkles.

One subject of the invention is thus a process for preparing a (cosmetic) composition for treating keratin materials, comprising a step of percolating a fluid at a pressure of at least 3 bar through at least one silicone or non-silicone conditioning agent, in solid or pasty form.

Another subject of the invention is a composition obtained via the process according to the invention.

A subject of the invention is also the use of the composition obtained according to the process of the invention, for the cosmetic treatment of keratin materials, and especially for conditioning the hair.

Another subject of the invention is a packaging device for performing the preparation process of the present invention.

Other subjects, characteristics, aspects and advantages of the invention will emerge even more clearly on reading the further nonlimiting description and the example that follows.

According to the invention, the process for preparing a (cosmetic) composition for treating keratin materials comprises a step of percolating fluid, preferably at a temperature of greater than or equal to 30° C., better still ranging from 30° C. to 150° C., and even more preferably ranging from 40° C. to 120° C., at a pressure of at least 3 bar (3×105 Pa), through at least one silicone or non-silicone conditioning agent as described below, which is preferably water-insoluble and non-cationic, in solid or pasty form.

Percolation is a movement of fluid through a porous medium, allowing the passage of the fluid due to the action or effect of pressure.

The fluid comprises at least steam optionally accompanied by liquid water and/or one or more cosmetically acceptable liquid and/or gaseous solvents. Preferably, the fluid is steam possibly accompanied by liquid water.

Examples of organic solvents that may be mentioned include C1-C4 lower alcohols, such as ethanol and isopropanol; polyols and polyol ethers, for instance 2-butoxyethanol, propylene glycol, propylene glycol monomethyl ether, diethylene glycol monomethyl ether and diethylene glycol monoethyl ether, and also aromatic alcohols, for instance benzyl alcohol or phenoxyethanol, and mixtures thereof.

The term “water-insoluble” means any compound which has a concentration of greater than or equal to 0.1% by weight in water at 25° C., does not form an isotropic solution that is transparent to the naked eye.

The conditioning agent is in solid or pasty form, preferably in solid form and even more preferably pulverulent.

For the purposes of the present invention, the term “pasty form” means a consistency intermediate between a solid phase and a liquid phase. The viscosity of this pasty phase is preferably greater than 0.1 Pa·s and even more preferably greater than 1 Pa·s, at 25° C. with a shear rate of 10 s-1.

The term “keratin materials” means the skin, the lips, and/or the integuments such as the nails, and keratin fibres, for example the eyelashes, the eyebrows and the hair.

The process of the present invention may be performed using a standard device for generating a pressurized fluid, at a temperature preferably of greater than or equal to 30° C., better still ranging from 30° C. to 150° C., and even more preferably ranging from 40° C. to 120° C. Such a device comprises a pressure-resistant chamber equipped with a thermal block, and also a circuit for conveying the fluid to the conditioning agent used in the invention.

According to another embodiment, the device additionally comprises a reservoir of liquid(s) and also a pump for conveying the liquid(s) to the chamber.

The liquid contained in the reservoir is either water, or a mixture of water and of one or more cosmetically acceptable solvents. Preferably, the liquid is water.

A device that is particularly useful for performing the process of the present invention is a coffee machine of the “espresso” type. Such machines are well known in the art. For example, these machines are described in patents AT 168 405, U.S. Pat. No. 2,688,911, DE 324 33 870 and IT 1 265 636.

According to one particular embodiment of the invention, the percolation step is performed with a fluid at a temperature of greater than or equal to 30° C., better still ranging from 30° C. to 150° C., and even more preferably ranging from 40° C. to 120° C., under a pressure ranging from 3 to 30 bar (3×105 to 3×106 Pa), preferably of at least 4 bar (4×105 Pa), more preferably greater than or equal to 10 bar (106 Pa) and most particularly ranging from 10 to 30 bar (106 to 3×106 Pa).

A (cosmetic) composition comprising at least one conditioning agent as described below, preferably in solid or pasty form, may be used directly in the device for generating the pressurized fluid in a container intended for this use. It may also be packaged in a particular packaging device, comprising a closed housing delimited by at least one wall that is at least partially permeable to fluid under a pressure of at least 3 bar, preferably from 3 to 30 bar, more preferably of at least 4 bar, better still greater than or equal to 10 bar and most particularly ranging from 10 to 30 bar. Such devices are described, for example, in patent applications WO 00/56629, EP 512 470, U.S. Pat. No. 5,897,899 or WO 99/03573. These packaging devices are generally protected against air, humidity and/or light.

According to one particular embodiment, the housing is delimited by two sealed sheets. According to another embodiment, the housing is delimited by a tray closed with a lid.

These devices may be manufactured from woven or nonwoven, plastic or plant materials, for example cellulose, metal such as aluminium, or composite materials. Such devices are described, for example, in patent applications WO 00/56629, EP 512 470, U.S. Pat. No. 5,897,899 or WO 99/03573.

The silicone or non-silicone conditioning agents used in the invention are not limited, and are preferably water-insoluble and/or non-cationic. They may be chosen, without limitation, from:
linear or branched fatty alcohols containing from 14 to 50 carbon atoms, such as cetyl alcohol, myristyl alcohol, stearyl alcohol, palmityl alcohol, behenyl alcohol and eicosanoyl alcohol;
linear or branched hydrocarbons of mineral or synthetic origin, such as paraffin waxes and derivatives thereof, poly(α-olefins), petroleum jelly, polydecenes and hydrogenated polyisobutenes;
waxes,
compounds of ceramide type, and
organomodified or non-organomodified silicone gums or resins.

Conditioning agents that are particularly suitable for the invention are waxes.

For the purpose of the present invention, a wax is a lipophilic fatty compound that is solid or pasty at room temperature (20 to 25° C.), with a reversible solid/liquid change of state, having a melting point of greater than 25° C., which may be up to 200° C., generally with a hardness of greater than 0.5 MPa for the solids and a hardness of between 0.001 and 0.5 MPa for the pastes, and having at the melting point an anisotropic crystal organisation.

As water-insoluble waxes that may be used in the present invention, mention may be made especially of waxes of animal, plant, mineral or synthetic origin, such as beeswax, spermaceti, fluoro and perfluoro waxes, lanolin wax, hydrogenated lanolin wax and acetylated lanolin wax; candelilla wax, ouricurry wax, carnauba wax, Japan wax, cocoa butter, cork fibre wax or sugarcane wax, rice bran wax, pine wax and cottonseed wax; microcrystalline waxes, paraffin wax, petrolatum, petroleum jelly, ozokerite and montan wax; hydrogenated oils with a melting point of greater than about 40° C., for instance hydrogenated jojoba oil; polyethylene waxes and the waxes obtained by Fischer-Tropsch synthesis.

For the purpose of the present invention, the term ‘compounds of ceramide type’ and ‘ceramide compounds’ includes ceramides and/or glycoceramides and/or pseudoceramides and/or neoceramides, which are natural or synthetic.

Compounds of ceramide type are described, for example, in patent applications DE 4 424 530, DE 4 424 533, DE 4 402 929, DE 4 420 736, WO 95/23807, EP-A-0 646 572, WO 95/16665, FR-2 673 179, EP-A-0 227 994, WO 94/07844, WO 94/24097 and WO 94/10131.

The compounds of ceramide type that may preferably be used according to the present invention correspond, for example, to the general formula (I) below: 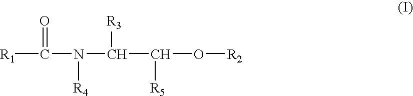
in which:
R1 denotes:
   either a saturated or unsaturated, linear or branched C1-C50, preferably C5-C50, hydrocarbon-based radical, it being possible for this radical to be substituted with one or more hydroxyl groups which may be esterified with an acid R7COOH, R7 being a saturated or unsaturated, linear or branched, optionally mono- or polyhydroxylated C1-C35 hydrocarbon-based radical, it being possible for the hydroxyl(s) of the radical R7 to be esterified with a saturated or unsaturated, linear or branched, optionally mono- or polyhydroxylated C1-C35 fatty acid,
   or a radical R1″-(NR^{o}-CO)-R1′-, R^{o} denoting a hydrogen atom or a mono- or polyhydroxylated, preferably monohydroxylated, C1-C20 hydrocarbon-based radical, R1′ and R1″ being hydrocarbon-based radicals, the sum of the carbon atoms of which is between 9 and 30, R1′ being a divalent radical,
   or a radical R8-O—CO—(CH2)ₚ—, R8 denoting a C1-C20 hydrocarbon-based radical, p being an integer ranging from 1 to 12;
R2 is chosen from a hydrogen atom, a radical of saccharide type, in particular a (glycosyl)n, (galactosyl)m or sulfogalactosyl radical, a sulfate or phosphate residue, a phosphorylethylamine radical or a phosphorylethylammonium radical, in which n is an integer ranging from 1 to 4 and m is an integer ranging from 1 to 8;
R3 denotes a hydrogen atom or a saturated or unsaturated, mono- or polyhydroxylated or non-hydroxylated C1-C33 hydrocarbon-based radical, it being possible for the hydroxyl(s) to be esterified with a mineral acid or an acid R7COOH, R7 having the same meanings as above, it being possible for the hydroxyl(s) to be etherified with a (glycosyl)ₙ, (galactosyl)ₘ, sulfogalactosyl, phosphorylethylamine or phosphorylethylammonium radical, it being also possible for R3 to be substituted with one or more C1-C14 alkyl radicals;
preferably, R3 denotes a C15-C26 α-hydroxyalkyl radical, the hydroxyl group optionally being esterified with a C16-C30 α-hydroxy acid;
R4 denotes a hydrogen atom, a methyl or ethyl radical, a saturated or unsaturated, linear or branched, optionally hydroxylated C3-C50 hydrocarbon-based radical or a radical —CH2—CHOH—CH2—O—R6 in which R6 denotes a C10-C26 hydrocarbon-based radical or a radical R8-O—CO—(CH2)ₚ—, R8 denoting a C1-C20 hydrocarbon-based radical, p being an integer ranging from 1 to 12;
R5 denotes a hydrogen atom or a saturated or unsaturated, linear or branched, optionally mono- or polyhydroxylated C1-C30 hydrocarbon-based radical, it being possible for the hydroxyl(s) to be etherified with a (glycosyl)ₙ, (galactosyl)ₘ, sulfogalactosyl, phosphorylethylamine or phosphorylethylammonium radical,
with the proviso that when R3 and R5 denote a hydrogen atom or when R3 denotes a hydrogen atom and R5 denotes a methyl radical, then R4 does not denote a hydrogen atom or a methyl or ethyl radical.

Among the compounds of formula (I) above, the ceramides and/or glycoceramides described by Downing in Journal of Lipid Research, Vol. 35, pages 2060-2068, 1994 or those described in French patent application FR-2 673 179 are preferred.

The compounds of ceramide type which are more particularly preferred according to the invention are the compounds of formula (I) for which R1 denotes a saturated or unsaturated, optionally hydroxylated alkyl radical derived from C14-C22 fatty acids; R2 denotes a hydrogen atom; and R3 denotes a linear, optionally hydroxylated C11-C17 and preferably C13-C15 radical.

Such compounds are, for example:
2-N-linoleoylaminooctadecane-1,3-diol,
2-N-oleoylaminooctadecane-1,3-diol,
2-N-palmitoylaminooctadecane-1,3-diol,
2-N-stearoylaminooctadecane-1,3-diol,
2-N-behenoylaminooctadecane-1,3-diol,
2-N-[2-hydroxypalmitoyl]aminooctadecane-1,3-diol,
2-N-stearoylaminooctadecane-1,3,4-triol and in particular N-stearoyl-phytosphingosine,
2-N-palmitoylaminohexadecane-1,3-diol
   or mixtures of these compounds.

The compounds of formula (I) for which R1 denotes a saturated or unsaturated alkyl radical derived from C12-C22 fatty acids; R2 denotes a galactosyl or sulfogalactosyl radical; and R3 denotes a saturated or unsaturated C12-C22 hydrocarbon-based radical and preferably a —CH(OH)—CH═CH—(CH2)12-CH3 group, may also be used.

By way of example, mention may be made of the product consisting of a mixture of glycoceramides, sold under the trade name Glycocer by the company Waitaki International Biosciences.

The compounds of formula (I) described in patent applications EP-A-0 227 994 and WO 94/07844 may also be used.

Such compounds are, for example Questamide H (bis(N-hydroxyethyl-N-cetyl)malonamide) sold by the company Quest.

N-Docosanoyl-N-methyl-D-glucamine described in patent application WO 94/24097 may also be used.

Other conditioning agents according to the invention are organomodified or non-organomodified silicone gums or resins.

The non-organomodified silicones (or polyorganosiloxanes) that may be used in the present invention include polysiloxanes comprising on the silicon atoms hydroxyl groups or alkyl, alkenyl (in particular vinyl) or aryl groups, these groups being substituted or unsubstituted. They preferably have a viscosity ranging from 50,000 to 750,000 m2/s at 25° C.

The organopolysiloxanes are defined in greater detail in Walter Noll's “Chemistry and Technology of Silicones” (1968) Academic Press.

Examples of non-organomodified silicone resins or gums that may be mentioned include:
(i) polydialkylsiloxanes;
(ii) polydiarylsiloxanes;
(iii) polyalkylarylsiloxanes;
(iv) or mixtures thereof.

The alkyl radicals especially contain from 1 to 10 carbon atoms and in particular denote methyl. The aryl radicals more particularly denote phenyl.

These silicones are more particularly chosen from polydialkylsiloxanes, among which mention may be made mainly of polydimethylsiloxanes containing trimethylsilyl or dimethylsilanol end groups.

Among the polydialkylsiloxanes that may mainly be mentioned are:
linear polydimethylsiloxanes containing trimethylsilyl end groups and having a viscosity from 50,000 to 750,000 mm2/s at 25° C., for instance, and in a non-limiting manner, the Silbione® silicones of the 70047 series sold by Rhodia Chimie, especially the oil Silbione® 70047 V 500 000 from Rhodia Chimie or the polydimethylsiloxane with a viscosity of 300,000 mm2/s (300,000 cSt), sold under the name DC200 Fluid 300 000 by the company Dow Corning, and silicones of the 200 series from the company Dow Corning, more particularly such as DC200 with a viscosity of 60,000 mm2/s (60,000 cSt);
linear polydimethylsiloxanes containing hydroxydimethylsilyl end groups (Dimethiconol according to the CTFA name).

In this category of polyalkylsiloxanes, mention may also be made of the polyalkylsiloxanes sold by the company Goldschmidt under the trade names Abil Wax® 9800 and Abil Wax® 9801, which are poly(C1-C20)alkylsiloxanes.

The polyalkylarylsiloxanes are particularly chosen from linear and/or branched polydimethyl-methylphenylsiloxanes and polydimethyl-diphenylsiloxanes with a viscosity of from 50,000 to 1,000,000 mm2/s at 25° C.

The silicone gums in accordance with the invention are more particularly polyorganosiloxanes with a number-average molecular weight of between 200,000 and 1,000,000.

Mention may be made, for example, of the following compounds:
polydimethylsiloxane,
poly[(dimethylsiloxane)/(methylvinylsiloxane)],
poly[(dimethylsiloxane)/(diphenylsiloxane)],
poly[(dimethylsiloxane)/(phenylmethylsiloxane)],
poly[(dimethylsiloxane)/(diphenylsiloxane)/(methylvinylsiloxane)].

A product that may more particularly be used in accordance with the invention is the SE 30 gum from General Electric, corresponding to a dimethicone with a number-average molecular weight of 500,000.

The organopolysiloxane resins that can be used in accordance with the invention are crosslinked siloxane compounds containing the following units:
R2SiO2/2, R3SiO1/2, RSiO3/2 and SiO4/2 in which R represents a hydrocarbon-based group containing 1 to 16 carbon atoms or a phenyl group. Among these products, those particularly preferred are the ones in which R denotes a C₁-C₄ lower alkyl radical, more particularly methyl, or a phenyl radical.

Among these resins, mention may be made of the product sold under the name “Dow Corning 593” or those sold under the names “Silicone Fluid SS 4230 and SS 4267” by the company General Electric, which are silicones of dimethyl/trimethyl siloxane structure.

Mention may also be made of the trimethyl siloxysilicate type resins sold in particular under the names X22-4914, X21-5034 and X21-5037 by the company Shin-Etsu.

The organomodified silicones that can be used in accordance with the invention are silicones as defined above and containing in their structure one or more organofunctional preferably non-cationic groups attached via a hydrocarbon-based group.

Among the organomodified silicones, mention may be made of polyorganosiloxanes comprising:
polyethyleneoxy and/or polypropyleneoxy groups optionally comprising C₆-C₂₄ alkyl groups;
thiol groups;
alkoxylated groups such as the products Abil Wax® 2428, 2434 and 2440 sold by the company Goldschmidt;
hydroxylated groups such as the polyorganosiloxanes containing a hydroxyalkyl function, described in French patent application FR-A-85/16334;
C3-C30 acyloxyalkyl groups;
anionic groups of carboxylic type, such as a behenate group;
polyester groups, such as polydimethylsiloxanes containing α,ω-hydroxypolycaprolactone groups and polydimethylsiloxanes-α,ω-silanols containing jasminate groups; and/or
hydroxyacylamino groups.

It is also possible to use as organomodified silicones in the process of the invention block copolymers containing a linear polysiloxane-polyoxyalkylene block of (A-B)ₙ type, preferably corresponding to the following general formula:
([Y(R2SiO)aR′2SiYO][CnH2nO)b])c (II)
in which:
R and R′, which may be identical or different, represent a monovalent hydrocarbon-based radical,
n is an integer ranging from 2 to 4,
a is an integer greater than or equal to 5, preferably between 5 and 200 and even more particularly between 5 and 100,
b is an integer greater than or equal to 4, preferably between 4 and 200 and even more particularly between 5 and 100,
c is an integer greater than or equal to 4, preferably between 4 and 1000 and even more particularly between 5 and 300,
Y represents a divalent organic group linked to the adjacent silicon atom via a carbon-silicon bond and to the polyoxyalkylene block via an oxygen atom,
the average molecular weight of each siloxane block is between about 400 and about 10,000, the average molecular weight of each polyoxyalkylene block being between about 300 and about 10,000,
the siloxane blocks represent from about 10% to about 95% by weight of the block copolymer,
the weight-average molecular weight of the block copolymer being at least 3,000, preferably between 5,000 and 1,000,000 and even more particularly between 10,000 and 200,000.

R and R′ are preferably chosen from the group comprising alkyl radicals, for instance methyl, ethyl, propyl, butyl, pentyl, hexyl, octyl, decyl and dodecyl radicals, aryl radicals, for instance phenyl and naphthyl, aralkyl radicals, for instance benzyl and phenylethyl, and tolyl, xylyl and cyclohexyl radicals.

Y is preferably —R″-, —R″-CO—, —R″-NHCO—, —R″-NHCO—NH—R′″-NHCO— or —R″-OCONH—R′″-NHCO—, in which R″ is a divalent alkylene group, for instance an ethylene, propylene or butylene group, and R′″ is a divalent alkylene group or a divalent arylene group, for instance —C6H4—, —C6H4—C6H4—, —C6H4—CH2—C6H4— or —C6H4—C(CH3)2C6H4—.

Even more preferably, Y represents a divalent alkylene radical, more particularly the —CH2—CH2-CH2— radical or the —C4H8— radical.

The preparation of the block copolymers used in the context of the present invention is described in European patent application EP 0 492 657 A1.

According to the invention, it is also possible to use, as organomodified silicone, silicone polyurethanes such as those described in patent applications EP 0 751 162, EP 0 619 111 and EP 1 025 833.

According to the invention, it is also possible to use organomodified silicones comprising a polysiloxane portion and a portion consisting of a nonsilicone organic chain, one of the two portions constituting the main chain of the polymer, and the other being grafted onto the main chain. These polymers are described, for example, in patent applications EP-A-0 412 704, EP-A-0 412 707, EP-A-0 640 105, WO 95/00578, EP-A-0 582 152 and WO 93/23009 and patents U.S. Pat. No. 4,693,935, U.S. Pat. No. 4,728,571 and U.S. Pat. No. 4,972,037.

These polymers are preferably anionic or nonionic.

Such polymers are, for example, copolymers that can be obtained by free radical polymerization from the monomer mixture formed from:
a) 50 to 90% by weight of tert-butyl acrylate;
b) 0 to 40% by weight of acrylic acid;
c) 5 to 40% by weight of a silicone macromer of formula: 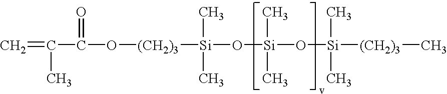
in which v is a number from 5 to 700; the weight percentages being calculated relative to the total weight of the monomers.

Other examples of grafted silicone polymers are, in particular, polydimethyl-siloxanes (PDMS) onto which are grafted, via a thiopropylene-type connecting chain, mixed polymer units of the poly(meth)acrylic acid type and of the polyalkyl (meth)acrylate type, and polydimethylsiloxanes (PDMS) onto which are grafted, via a thiopropylene-type connecting chain, polymer units of the polyisobutyl (meth)acrylate type.

The polyorganosiloxanes that are particularly preferred in accordance with the invention are:
silicones chosen from the polydimethylsiloxane family containing trimethylsilyl end groups, such as the oils of the DC200 series from Dow Corning, with a viscosity of 60,000 mm2/s (60,000 cSt), of the Silbione® 70047 and 47 series, and more particularly the oil Silbione® 70 047 V 500 000 sold by the company Rhodia Chimie, or the silicone oil AK 300 000 from the company Wacker, polydimethylsiloxanes containing dimethylsilanol end groups such as the dimethiconols, or polyalkylarylsiloxanes such as the oil Silbione 70641 V 200 sold by the company Rhône-Poulenc; and
the organopolysiloxane resin sold under the name Dow Corning 593.

The conditioning agent of the invention may be used as a mixture with one or more solid or pasty, and preferably pulverulent, adjuvants. The adjuvants may be chosen from clays, salts, anionic, nonionic, cationic or zwitterionic surfactants, natural or synthetic thickeners, optionally modified starch, glass beads, silica, Nylon, alumina, titanium dioxide, zeolites, polymethyl methacrylate (PMMA), chitosan, maltodextrin, cyclodextrin, mono- or disaccharides, for instance glucose, sucrose, sorbitol and fructose, zinc oxide, zirconium oxide, silica beads, talc, polyaspartic acid, borosilicates, especially calcium borosilicate, polyethylene, cotton, polytetrafluoroethylene (PTFE), cellulose and its derivatives, superabsorbent compounds, magnesium carbonate, calcium carbonate, corn seeds, polyacrylamide, porous hydroxyapatite, silk, collagen, sawdust, wrack powder, meals or extracts of wheat, rice, pea, lupin, soybean or barley, crosslinked polyvinylpyrrolidone, calcium alginate, active charcoal, and poly(vinylidene chloride/acrylonitrile) particles, especially those sold under the general name “Expancel®” by the company Akzo Nobel under the particular reference “Expancel® WE” or “Expancel DE”, and mixtures thereof.

When one or more adjuvants are present, the conditioning agent(s) of the invention is (are) preferably present in an amount ranging from 0.5% to 99% by weight, better still from 1% to 80% by weight and even more preferably from 2% to 60% by weight relative to the total weight of conditioning agent(s) and adjuvant(s) in solid or pasty form.

When plants or plant extracts are used in the process of the present invention, they may be pretreated before the percolation step. The pretreatment can be drying, roasting, cryogrinding or freeze-drying.

The (cosmetic) composition for treating keratin materials obtained according to the process of the invention can contain, besides the conditioning agent(s) and the component(s) of the fluid, i.e. water and/or cosmetically acceptable solvent(s), optionally all or some of the adjuvant(s) present in the solid or pasty mixture.

The invention also relates to a composition obtainable via the process according to the invention, the particularly preferred composition being free of preserving agents. Using the preparation process of the invention, a (cosmetic) composition for treating keratin materials is obtained, which may be applied directly to keratin materials, or which may be mixed with a cosmetically acceptable medium, or alternatively at least one additive conventionally used in cosmetics may be added thereto by an operator. At least two compositions obtained via the process of the invention may also be mixed together. The (cosmetic) composition for treating keratin materials optionally resulting from the mixture(s) and/or addition(s) indicated above will be referred to hereinbelow as the final cosmetic treatment composition or final composition.

One particular embodiment of the invention comprises applying the composition obtained by means of a device not requiring any human intervention, and optionally equipped with a cooling means.

The amount of the conditioning agent(s) present in the final cosmetic treatment composition is generally between 0.001% and 50% by weight approximately, preferably between 0.005% and 30% by weight, and even more preferably between 0.01% and 20% by weight, relative to the total weight of the final cosmetic treatment composition.

When the cosmetic treatment composition obtained via the process of the present invention is mixed with a cosmetically acceptable medium, such a medium generally consists of water or of a mixture of water and of at least one organic solvent to dissolve the compounds that would not be sufficiently soluble in water.

The term “cosmetically acceptable” means a medium which is compatible with keratin materials and especially the skin, the lips and/or the integuments, and which, in addition, has an appearance, a feel, a smell and optionally a flavour which are pleasing to the user.

Examples of organic solvents that may be mentioned include C1-C4 lower alcohols, such as ethanol and isopropanol; polyols and polyol ethers, for instance 2-butoxyethanol, propylene glycol, propylene glycol monomethyl ether and diethylene glycol monomethyl ether and diethylene glycol monoethyl ether, and also aromatic alcohols, for instance benzyl alcohol or phenoxyethanol, and mixtures thereof.

The solvents are preferably present in proportions preferably of between 1% and 40% by weight approximately and even more preferably between 5% and 30% by weight approximately relative to the total weight of the final cosmetic treatment composition.

At least one additive conventionally used in cosmetics may also be added to the cosmetic treatment compositions obtained according to the process of the present invention. Examples of such additives that may be mentioned include anionic, cationic, nonionic, amphoteric or zwitterionic surfactants, or mixtures thereof; anionic, cationic, nonionic, amphoteric or zwitterionic polymers, or mixtures thereof; mineral or organic thickeners, and in particular anionic, cationic, nonionic and amphoteric polymeric associative thickeners; antioxidants; penetrating agents; sequestering agents; fragrances; buffers; dispersants; conditioning agents other than those described above, for instance silicone oils; film-forming agents; preserving agents; opacifiers, and nacreous or coloured pigments.

The above additives are generally present in an amount for each of them of between 0.01% and 20% by weight relative to the weight of the final composition.

Needless to say, a person skilled in the art will take care to select this or these optional complementary compounds such that the advantageous properties intrinsically associated with the cosmetic treatment composition in accordance with the invention are not, or are not substantially, adversely affected by the addition(s) envisaged.

The pH of the final cosmetic treatment composition is generally between 3 and 12 approximately, and preferably between 5 and 11 approximately. It may be adjusted to the desired value using acidifying or basifying agents usually used in cosmetics, or alternatively using standard buffer systems.

Among the acidifying agents that may be mentioned, for example, are mineral or organic acids such as hydrochloric acid, orthophosphoric acid, sulfuric acid, carboxylic acids such as acetic acid, tartaric acid, citric acid and lactic acid, and sulfonic acids.

Among the basifying agents that may be mentioned, for example, are ammonia, alkaline carbonates, alkanolamines such as mono-, di- and triethanolamine and derivatives thereof, sodium hydroxide, potassium hydroxide and the compounds of the following formula: 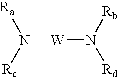
in which W is a propylene residue that is optionally substituted with a hydroxyl group or a C1-C4 alkyl radical; Ra, Rb, Rc and Rd, which may be identical or different, represent a hydrogen atom, a C1-C4 alkyl radical or a C1-C4 hydroxyalkyl radical.

The final cosmetic treatment composition may be in any of various forms, such as in the form of liquids, creams or gels, or in any other form that is suitable for treating keratin materials, and especially human hair.

The final cosmetic treatment composition may be used, for example, as a shampoo, a rinse-out or leave-in conditioner, a deep-down care mask, a shower gel, or a lotion or cream for treating keratin materials.

The present invention also relates to a cosmetic process for treating keratin materials, comprising the preparation of a cosmetic treatment composition according to the process as defined above, and its application to the keratin materials, for example by means of an operator or by means of a device not requiring any human intervention. The application time may range for example between 15 seconds and one hour.

Before application, the cosmetic treatment composition obtained according to the process of the invention may be mixed with a cosmetically acceptable medium and/or with one or more additives conventionally used in cosmetics, as described above.

Another embodiment comprises preparing at least two cosmetic treatment compositions according to the process of the invention, mixing them together, and optionally adding a cosmetically acceptable medium and/or one or more additives conventionally used in cosmetics, as described above, and then in applying the final composition obtained to keratin materials.

The example below illustrates the present invention, but does not limit it.

### EXAMPLE

The solid ingredients below are mixed together in the proportions indicated as weight percentages relative to the total weight of solid mixture:

| | |
|---|---|
| Associative cellulosic cationic polymer sold under | 8% |
| the trade name Quatrisoft LM 200 by the company Amerchol | |
| Mixture of linear alcohols (C18/C20/C22) sold | 4% |
| under the trade name Nafol 1822 by the company Sasol | |
| Sodium lauroyl sarcosinate | 80% |
| Polydimethylsiloxane containing behenate group | 8% |
| sold under the trade name Mirasil Wax-B by the company Rhodia | |

5 g of this mixture are placed in a commercial espresso machine. Steam is then passed through until a composition (A) having a final volume of 50 ml is obtained.

A cosmetic treatment composition ready to be applied to the hair is thus obtained.

Soft hair and a good styling and fixing effect are obtained.

Two parts by weight of composition (A) can be added to one part by weight of an aqueous composition(B) containing 1% by weight of hydroxyethylcellulose, in order to facilitate application.

The above written description of the invention provides a manner and process of making and using it such that any person skilled in this art is enabled to make and use the same, this enablement being provided in particular for the subject matter of the appended claims, which make up a part of the original description and including a process for preparing a composition, comprising percolating fluid comprising at least steam, at a pressure ranging from 3 to 30 bar, through at least one silicone or non-silicone conditioning agent, in solid or pasty form, chosen for example from linear or branched fatty alcohols comprising from 14 to 50 carbon atoms, linear or branched hydrocarbons, waxes, ceramide compounds, organomodified or non-organomodified silicone gums or resins, and mixtures thereof, the compositon prepared, and a process using the composition for treating keratin fibers.

As used herein, the phrases “selected from the group consisting of,” “chosen from,” “selected from,” and the like include mixtures of the specified materials.

Where compounds are described as, e.g, “cellulose and its derivatives” or “compounds of the cellulose type” an alternate is “cellulose and cellulose compounds” where cellulose derivatives and compounds share a common core/structure with cellulose. One of ordinary skill in the art knows how to identify derivatives and compounds of compound X based on the structure of X and the similarity in structure of the derivatives and compounds. Thus, the term “compounds of ceramide type” and “ceramide compounds” include ceramide and ceramide compounds.

Where a term is enclosed by parentheses it is an optional modifier. For example, the term “(cosmetic) composition” describes both a composition in general and a cosmetic composition.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A process for preparing a composition, comprising percolating fluid comprising at least steam, at a pressure ranging from 3 to 30 bar, through at least one silicone or non-silicone conditioning agent, in solid or pasty form, chosen from linear or branched fatty alcohols comprising from 14 to 50 carbon atoms, linear or branched hydrocarbons, waxes, ceramide compounds, organomodified or non-organomodified silicone gums, organomodified or non-organomodified silicone resins, and mixtures thereof.

2. The process according to claim 1, wherein the fluid further comprises at least one of liquid water and a cosmetically acceptable liquid and/or gaseous solvent.

3. The process according to claim 1, comprising percolating fluid comprising at least steam, at a pressure ranging from 3 to 30 bar, through at least one wax, wherein the at least one wax is chosen from beeswax, spermaceti, fluoro and perfluoro waxes, lanolin wax, hydrogenated lanolin wax and acetylated lanolin wax; candelilla wax, ouricurry wax, carnauba wax, Japan wax, cocoa butter, cork fibre wax or sugarcane wax, rice bran wax, pine wax and cottonseed wax; microcrystalline waxes, paraffin wax, petrolatum, petroleum jelly, ozokerite and montan wax; hydrogenated oils with a melting point of greater than 40° C.; polyethylene waxes and the waxes obtained by Fischer-Tropsch synthesis.

4. The process according to claim 1, comprising percolating fluid comprising at least steam, at a pressure ranging from 3 to 30 bar, through at least one ceramide compound, wherein the at least one ceramide compound is chosen from:
2-N-linoleoylaminooctadecane-1,3-diol,
2-N-oleoylaminooctadecane-1,3-diol,
2-N-palmitoylaminooctadecane-1,3-diol,
2-N-stearoylaminooctadecane-1,3-diol,
2-N-behenoylaminooctadecane-1,3-diol,
2-N-[2-hydroxypalmitoyl]aminooctadecane-1,3-diol,
2-N-stearoylaminooctadecane-1,3,4-triol,
2-N-palmitoylaminohexadecane-1,3-diol, and
N-docosanoyl-N-methyl-D-glucamine.

5. The process according to claim 1, comprising percolating fluid comprising at least steam, at a pressure ranging from 3 to 30 bar, through at least one non-organomodified silicone gum or resin, wherein the at least one non-organomodified silicone gum or resin is chosen from polydialkylsiloxanes, polydiarylsiloxanes and polyalkylarylsiloxanes.

6. The process according to claim 5, comprising percolating fluid comprising at least steam, at a pressure ranging from 3 to 30 bar, through at least one silicone resin chosen from crosslinked siloxane compounds containing the units:
R2SiO2/2, R3SiO1/2, RSiO3/2 and SiO4/2
in which R represents a hydrocarbon-based group containing 1 to 16 carbon atoms or a phenyl group.

7. The process according to claim 1, comprising percolating fluid comprising at least steam, at a pressure ranging from 3 to 30 bar, through at least one organomodified silicone chosen from polyorganosiloxanes comprising polyethyleneoxy and/or polypropyleneoxy groups, thiol groups, hydroxylated groups, acyloxyalkyl groups, anionic groups of the carboxylic type, polyester groups and/or hydroxyacylamino groups.

8. The process according to claim 1, wherein the conditioning agent in solid or pasty form is present as a mixture with at least one adjuvant.

9. The process according to claim 8, wherein the adjuvant is chosen from clays, salts, anionic, non-ionic, cationic or zwitterionic surfactants, natural or synthetic thickeners, optionally modified starch, glass beads, silica, Nylon, alumina, titanium dioxide, zeolites, polymethyl methacrylate (PMMA), chitosan, maltodextrin, cyclodextrin, mono- or disaccharides, zinc oxide, zirconium oxide, silica beads, talc, polyaspartic acid, borosilicates, especially calcium borosilicate, polyethylene, cotton, polytetrafluoroethylene (PTFE), cellulose and its derivatives, superabsorbent compounds, magnesium carbonate, calcium carbonate, corn seeds, polyacrylamide, porous hydroxyapatite, silk, collagen, sawdust, wrack powder, extracts of wheat, rice, pea, lupin, soybean or barley, crosslinked polyvinylpyrrolidone, calcium alginate, active charcoal, poly(vinylidene chloride/acrylonitrile) particles and mixtures thereof.

10. The process according to claim 8, wherein the conditioning agent is present in an amount of 0.5% to 99% by weight relative to the total weight of conditioning agent(s) and adjuvants in solid or pasty form.

11. The process according to claim 1, wherein the percolation step is performed with a fluid at a pressure of at least 10 bar.

12. A composition obtained by percolating fluid comprising at least steam, at a pressure ranging from 3 to 30 bar, through at least one silicone or non-silicone conditioning agent, in solid or pasty form, chosen from linear or branched fatty alcohols comprising from 14 to 50 carbon atoms, linear or branched hydrocarbons, waxes, ceramide compounds, organomodified or non-organomodified silicone gums, organomodified or non-organomodified silicone resins, and mixtures thereof.

13. The composition according to claim 12, which does not comprise preserving agent.

14. The composition according to claim 12, wherein the conditioning agent in solid or pasty form is present as a mixture with at least one adjuvant, and wherein the composition obtained comprises, besides the conditioning agent(s) and the component(s) of the fluid, all or some of the adjuvant(s) present in the mixture in solid or pasty form.

15. A process for treating keratin material, wherein the composition of claim 12 is applied to keratin material.

16. The process according to claim 15, wherein the composition is applied to the keratin material by a device not requiring any human intervention.

17. The process according to claim 15, wherein, before application, the composition is mixed with a cosmetically acceptable medium and/or with one or more additives used in cosmetics.

18. The process according to claim 15, wherein, before application, the composition is mixed with another, different such composition and the mixture is applied to the keratin material.

19. A device for packaging a composition, comprising a closed housing delimited by at least one wall that is at least partially permeable to fluid at a pressure of at least 3 bar, the composition containing at least one silicone or non-silicone conditioning agent, in solid or pasty form.

20. The device according to claim 19, wherein the housing is delimited by two sealed sheets or by a tray closed with a lid.
